(19) United States
(12) Patent Application Publication
(10) Pub. No.: US 2013/0092159 A1
(43) Pub. Date: Apr. 18, 2013

(54) METHOD AND DEVICE FOR SUPPLYING AT LEAST ONE MEDICAL GAS TO A PATIENT RECEIVING ARTIFICIAL RESPIRATION WITH THE AID OF A VENTILATOR

(71) Applicants: Hermann Ulrichskötter, Freiburg (DE); Rainer Köbrich, Neulussheim (DE)

(72) Inventors: Hermann Ulrichskötter, Freiburg (DE); Rainer Köbrich, Neulussheim (DE)

(73) Assignee: MAQUET VERTRIEB UND SERVICE DEUTSCHLAND GMBH, Rastatt (DE)

(21) Appl. No.: 13/643,879

(22) PCT Filed: Nov. 30, 2010
(86) PCT No.: PCT/EP2010/068557

§ 371 Date: Dec. 21, 2012

(30) Foreign Application Priority Data
Apr. 29, 2010 (DE) 10 2010 016 699.5

(51) Int. Cl. A61M 16/12; A61M 16/10; A61M 16/00

(52) U.S. Cl. CPC A61M 16/12; A61M 16/0057; A61M 16/104
USPC 128/203.14

(57) ABSTRACT

A device and method for administering at least one medical gas (NO) to a patient mechanically ventilated by means of a ventilator. The ventilator produces a constant respiratory gas flow (O₂/N₂) in a feed line. Gas pulses of the medical gas (NO) are supplied to said respiratory gas flow. The gas pulses are produced by means of at least two regulating means arranged in parallel and are fed to the line. Here, a control unit controls the regulating means depending on at least one parameter of the respiration of the patient.

### CROSS REFERENCE TO RELATED APPLICATIONS

This application is entitled to the benefit of and incorporates by reference essential subject matter disclosed in International Patent Application No. PCT/EP2010/068557 filed on Nov. 30, 2010 and German Patent Application No. 10 2010 016 699.5 filed Apr. 29, 2010.

### FIELD OF THE INVENTION

The invention relates to a method and a device for administering at least one medical gas to a mechanically ventilated patient. A machine ventilator produces a respiratory gas flow in at least a portion of a line supplying respiratory gas. A predetermined amount of a medical gas to be administered is added to this respiratory gas flow. The gas mixture provided by the respiratory gas flow of the ventilator and the medical gas added to this flow are supplied to a connecting piece, such as a so-called Y-piece from which a patient feed line leads to the mechanically ventilated patient and from which a further line branches off. Via this further line at least the gas exhaled by the patient and the proportion of the respiratory gas introduced into the first line by the ventilator and the medical gas fed into the first line which have not been inhaled by the patient are discharged via a second line.

### BACKGROUND OF THE INVENTION

Documents EP 0 937 479 B1, EP 0 937 479 B1, U.S. Pat. No. 5,558,083, EP 0 786 264 B1, EP 1 516 639 B1, and EP 0 723 466 B1 disclose devices and methods for delivering nitrogen monoxide in a continuous and pulsed manner over the course of time to a mechanically ventilated patient. Control valves for setting the amount of nitrogen monoxide are provided, which valves, however, as a function of the design in each case, allow a defined amount of gas to pass through per unit time under specific, defined pressure conditions. Therefore, there is reliance on providing the medical gas in a combination appropriate for the device and on providing the patient with an amount of gas appropriate for the treatment in the opened state of the regulating means. However, it is desirable to wean the mechanically ventilated patient, if necessary, from the active ingredients of the medical gas in a continuous or stepwise manner and to reduce the amount of the administered medical gas per unit time. In the case of high, gas source-provided concentrations of the medical gas and in the case of a very low amount of gas to be supplied, a precise metering of low amounts of gas is therefore necessary, whereas in the case of gas sources having a low concentration of the medical gas and administration of relatively large amounts of the medical gas by means of the regulating means, substantially larger amounts of gas have to be introduced into the first line.

### SUMMARY OF THE INVENTION

It is an object of the invention to specify a method and a device for administering at least one medical gas to a patient mechanically ventilated by means of a ventilator, in which method and device the amount of gas to be administered is easily adjustable.

This object is achieved by a method having the features of claim 1 a method for administering at least one medical gas to a patient mechanically ventilated by means of a ventilator, in which a first end of a first line supplying at least respiratory gas from the ventilator, a first end of a second line discharging at least exhaled gas from the patient and a first end of a patient feed line are connected to one another via at least one connecting piece, by means of the ventilator at least in one portion of the first line a respiratory gas flow is produced, and in which the medical gas is introduced into the first line supplying the respiratory gas, characterized in that a gas source for providing the medical gas to be administered and the first line are connected via at least two regulating means arranged in parallel, wherein a connection is established between the gas source and the first line via each regulating means in the opened state, in that multiple gas pulses of the medical gas are fed successively into the first line (24) by means of the regulating means, and in that the gas pulses depending on at least one parameter of the respiration of the patient are fed and by a device having the features of the independent device claim. Advantageous developments of the invention are specified in the dependent claims.

What is achieved by the method and the device for administering at least one medical gas to a patient mechanically ventilated by means of a ventilator is that, via each of the two regulating means arranged in parallel, the medical gas can be introduced into the ventilator-produced constant respiratory gas flow in the first line and can thus be supplied to the patient. By opening one of the regulating means or both regulating means to produce the gas pulses, it is possible to set the amount, more particularly the volume, of the medical gas introduced into the first line with an appropriate selection of the pulse length and pulse repetition. In this case, the gas pulses, in particular depending on at least one parameter of the respiration of the patient are given. By this, it is especially achieved that the medical gas is supplied to the patient during an inhalation phase.

By appropriately selecting the dimensions of the regulating means, gas sources containing different concentrations of the medical gas can thus also be used, without requiring structural modifications of the device for administering the medical gas. Thus, both the method and the device provide variable adjustment of the amount of gas to be administered in large adjustment ranges and thus a broad concentration spectrum. The pulse-shaped partial pressure brought about by the gas pulses is measurable into the airways of the mechanically ventilated patient.

In an advantageous embodiment of the invention the flow rate and/or the gradient of the flow rate of the respiratory gas flow are used as parameters of the respiration. The respective pulse duration of the gas pulse and/or the respective volume flow of the medical gas during the gas pulses and/or the interval between consecutive gas pulses are controlled depending on the flow rate. The control is in particular such that the amount of the administered medical gas is proportional to the flow rate.

It is especially advantageous if a pulse frequency of the gas pulses, at least during a period of the inhalation phase is 26, 52, 104 or 208 pulses per minute. In case of such pulse sequences it could be observed that the periodic noise generation by the device was tolerated especially well by the patient and that the patient showed a good reaction to the application of the medical gas. As already mentioned, the gas injection effected by the gas pulses leads very quickly to a homogeneous partial pressure situation. It is advantageous if the amount of the medical gas supplied per gas pulse is different at least in case of two gas pulses during the inhalation phase. The amount of medical gas can in particular be set via the pulse duration and/or the flow volume during the gas pulse.

Further, it is advantageous when the regulating means are controlled by means of a or the control unit such that an amount of gas defined in relation to a gas pulse and/or gas volume defined in relation to a gas pulse is fed into the first line. As a result, the amount of gas or the gas volume that is required for the administration can be introduced into the first line in a simple manner.

It is particularly advantageous when the medical gas contains NO (nitrogen monoxide). The medical gas can in particular be provided as a gas mixture composed of NO (nitrogen monoxide) and N₂ (nitrogen). A gas mixture composed of NO (nitrogen monoxide) and He (helium) has also been found to be particularly advantageous, since especially helium can achieve particularly short reaction and response times. As a result, effective administration is possible especially in the case of newborn babies and in the case of premature babies and the relatively low amounts of the mixture composed of respiratory gas and medical gas that are inhaled by these patients.

It is further advantageous to have more than two regulating means arranged in parallel. Experiments have shown that it is particularly advantageous to have four regulating means arranged in parallel, wherein the regulating means are formed such that at least two of the regulating means in the opened state allow a different amount of gas to pass through. The regulating means arranged in parallel are preferably valves and are then also referred to as a valve bank. In this connection, it has been found to be particularly advantageous when, under the defined pressure conditions, the first valve has a flow of 0.16 liters per minute, valve 2 has a flow of 1.6 liters per minute and valves 3 and 4 each have a flow of 8 liters per minute when opened constantly (measured using medical air). It is further advantageous when regulating means are used which have a shortest realizable opening time of milliseconds, preferably in the range from 4 milliseconds to 7 milliseconds. The control unit can open the valves individually or in any desired combination, and so, in the case of the specific exemplary embodiments, a maximum flow of 17.76 liters per minute is possible.

It is particularly advantageous when a control unit optimizes the opening of the regulating means to the effect that a very long opening time is achieved within one cycle time of for example 104 gas pulses per minute. As a result, a constant as well as homogeneous injection of the medical gas into the respiratory air supplied to the patient is achieved. Also achieved as a result is a large adjustable metering range of the medical gas to be administered to the patient.

In one embodiment, 52×18.60 microliters of the medical gas are administered when only one valve having a flow of 0.16 liters per minute, 7 milliseconds opening time per gas pulse and a pulse frequency of 52 pulses per minute theoretically, is opened. However, owing to the required valve stroke and/or the response delay, 13 microliters are administered in practical experiments using these parameters. Even in the case of a premature baby, which has a tidal volume of 2.4 litres per minute, it is possible as a result to set a low concentration of 0.1 ppm with a starting concentration of the medical gas of 1000 ppm. As a result, after a more highly concentrated administration of the medical gas, it can be reduced in a stepwise or continuous manner to approximately 676 microliters per minute, and weaning of the patient from the medical gas or from the active ingredient thereof is therefore easily possible. Furthermore, the use of multiple regulating means connected in parallel makes it possible, at the administered concentrations, i.e., target concentrations, which are currently conventional, to also use more highly concentrated supply gas sources, with the result that said supply gas sources, more particularly supply gas cylinders, have to be exchanged at greater intervals, and as a result logistics and consumption costs can be lowered. Alternatively or additionally, the invention makes a larger therapeutic concentration spectrum clinically available.

As already mentioned, it is advantageous when the regulating means in an opened state allow volume flows differing face to face to pass through from the gas source to the first line. In the case of more than two regulating means, it is advantageous when at least two of the regulating means in the opened state allow different volume flows to pass through from the gas source to the first line. As a result, a concentration from a relatively large concentration spectrum can be set in a simple manner.

It is particularly advantageous when the regulating means each comprise at least one solenoid valve. Furthermore, a restricting orifice or another restricting means for limiting the volume flow flowing through the regulating means can be arranged upstream and/or downstream of at least one regulating means. Solenoid valves are, firstly, inexpensive and, secondly, solenoid valves have relatively short response times. The solenoid valves are controlled in particular in a binary manner, and so they are completely closed in a first operating state and completely opened in a second operating state. By means of the restricting means for limiting the volume flow flowing through the regulating means, it is possible to use regulating means of the same type, more particularly solenoid valves of the same type, wherein the volume flow flowing through the regulating means in the opened state differs owing to the provision of different flow resistances. As a result, it is easily possible to produce different volume flows through the regulating means.

In an advantageous development of the invention, gas is removed from the patient feed line. At least the proportion of the medical gas and/or the proportion of a reaction product of the medical gas in the removed gas is determined. The gas can be removed from the patient feed line via a measurement line and supplied to an analysis unit for the detection of at least the proportion of the medical gas and/or the proportion of a reaction product of the medical gas. More particularly, the removal and detection can be carried out once or more than once during one act of inhalation, preferably repeatedly during each act of inhalation. As a result, the concentration of the medical gas in the inhalation air can be easily determined, monitored and/or regulated. The inner diameter of the measurement line is preferably smaller than the diameter of the first line, the second line and the patient feed line.

It is further advantageous to compare the determined proportion of the medical gas, as the actual value, with a target value and, in the event of a determined deviation of the actual value from the preset target value, to adapt the amount of the medical gas introduced into the first line during each gas pulse as a function of the comparative result. Preferably, the proportion of the medical gas in the inhalation gas is regulated to the preset target value. As a result, the amount of the medical gas to be administered to the patient can be easily monitored, and/or kept constant. If, in addition to or as an alternative to the proportion of the medical gas, the proportion of a reaction product of the medical gas is analyzed, it is advantageous to determine the proportion of an oxidation product of the medical gas. If nitrogen monoxide (NO) is used as medical gas, the proportion of the oxidation product nitrogen dioxide (NO₂) can be determined in particular. The proportion of the determined nitrogen dioxide can then be compared with a permissible target value. When the target value is exceeded, the feeding of the medical gas into the first line can then be stopped or the volume of the fed medical gas can be reduced. In the event of an excessively high concentration of nitrogen dioxide in the mechanical ventilation gas, the patient can be harmed, and so this must be avoided.

It is further advantageous when the ventilator determines information concerning a flow profile of the respiration of the mechanically ventilated patient. Depending on the determined flow profile the control unit can then control the regulating means such that during the inhalation phases of the patient during each generated gas pulse they apply a larger amount of the medical gas into the first line and/or supply the gas pulses with a higher pulse frequency into the first line than is the case during the exhalation phases of the patient.

In an escecially preferred embodiment the same pulse frequence is used during the inhalation phase and during the respiration phase. Preferably, the pulse frequency is preset to 104 gas pulses per minute. Alternatively, during the inhalation phase the pulse frequency can be higher than during the exhalation phase. In case of an increased pulse frequency the volume supplied with each gas pulse can equal or be higher than the gas volume of the gas pulses during the exhalation phase.

Further, between the inhalation phase and the exhalation phase a standstill of the flow can be detected, and during this standstill of the flow no medical gas can be supplied. Moreover, preferably during the inhalation phase gas pulses having a larger pulse width than during the exhalation phase are introduced.

The solenoid valves used are preferably valves switchable between a completely closed and a completely opened position, which valves are controlled in a binary manner.

The invention can be used especially in neonatology for treating pulmonary hypertension of a premature baby with nitrogen monoxide. Nitrogen monoxide is also administered in order to treat patients after organ transplantations. However, the invention can also be used for administering other gaseous medicaments.

Depending on the clinical use, up to 10% of the inspired volume can originate from a gas source for providing gaseous medicaments. Such a gas source is also referred to as an additive gas source, since it is provided in addition to a respiratory gas source or oxygen source. The invention avoids the disadvantage in the prior art that a delay time arises from the time of measuring the flow velocity of the respiratory gas used for the inspiration of the patient up to the mechanical adjustment of a control valve used for feeding the medical gas, and that there is an occurrence of relatively large concentration fluctuations of the administered medical gas in the mechanically ventilated air provided to the patient with dynamic flow profiles. Furthermore, in the case of known control valves, the control range of the conducted medical gas is limited relatively strongly. In the case of valves which allow a large flow of the medical gas, low flow rates can only be set relatively imprecisely. In a further embodiment of the invention, discontinuous feeding by means of multiple gas pulses into the respiratory air of the ventilator patient circuit comprising the first line, the second line and the patient feed line can be carried out.

Further features and advantages of the invention are found in the following description, which more particularly elucidates the invention by means of exemplary embodiments in conjunction with the attached figures.

### BRIEF DESCRIPTION OF THE DRAWINGS

The following is shown:

FIG. 1 a diagram of a device for administering at least one medical gas to a patient mechanically ventilated by means of a ventilator according to a first exemplary embodiment;

FIG. 2 a diagram of components of an administering apparatus for administering the medical gas;

FIG. 3 a diagram of a device for administering at least one medical gas to a patient mechanically ventilated by means of a ventilator according to a second exemplary embodiment of the invention;

FIG. 4 a representation of the temporal course of the respiration of the mechanically ventilated patient and of the administration of the medical gas according to the first and second exemplary embodiment of the invention;

FIG. 5 a diagram of a device for administering at least one medical gas to a patient mechanically ventilated by means of a ventilator according to a third exemplary embodiment of the invention;

FIG. 6 a representation of the temporal course of the respiration of a mechanically ventilated patient and of the administration of the medical gas according to the third exemplary embodiment of the invention, and

FIG. 7 a representation of the temporal course of the respiration of a mechanically ventilated patient and of the administration of the medical gas according to a fourth exemplary embodiment of the invention;

### DETAILED DESCRIPTION OF THE PREFERRED EMBODIMENTS

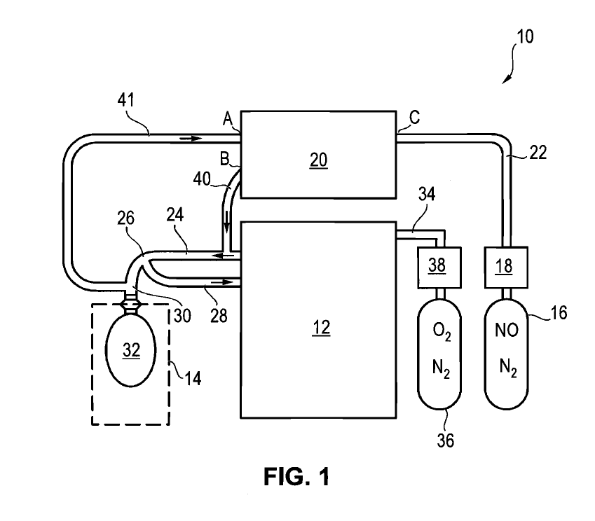

FIG. 1 shows a diagram of a device 10 for administering at least one medical gas to a patient 14 mechanically ventilated by means of a ventilator 12 according to a first exemplary embodiment of the invention. In this exemplary embodiment, the medical gas used is NO (nitrogen monoxide). This gas is provided in a gas cylinder 16 as a gas mixture (NO/N₂) comprising N₂ nitrogen and NO nitrogen monoxide. By means of a pressure regulator 18, the gas mixture NO/N₂ is supplied to a metering device 20 via a connecting tube 22 having a target pressure, preset at the pressure regulator 18, at the connector C of the metering device 20. From the ventilator 12, a first line 24 designed as a respiratory air tube leads to a connecting element 26 designed as a Y-piece. In addition, a second line 28 designed as a waste air tube and a patient feed line 30 are connected to the connecting element 26. In the present exemplary embodiment, the patient feed line 30 is connected to a test lung, simulating the patient 14, in the form of an inflatable balloon 32. To mechanically ventilate a living patient 14, the end of the patient feed line 30 leading to the patient 14 is connected to a face mask or to a tube inserted into the airways of the patient 14. The waste air tube 28 is led back to the ventilator 12, wherein the gas mixture flowing back through the waste air tube 28 is either discharged or recycled in the ventilator 12. In the present exemplary embodiment, the ventilator 12 is connected to a gas source in the form of a gas cylinder 36 via a connecting tube 34. The gas cylinder 36 contains a gas mixture (O₂/N₂) comprising oxygen (O₂) and nitrogen (N₂). The gas mixture O₂/N₂ is limited to a preset target value by means of a pressure regulator 38 and supplied to the ventilator 12 via the connecting tube 34. In other exemplary embodiments, oxygen and nitrogen can also be provided by means of separate gas sources 36, more particularly also via a central gas supply in a hospital.

The ventilator 12 produces a constant flow of respiratory gas in the respiratory air tube 24. The medical gas mixture NO/N₂ determined for the treatment of the patient is supplied to this constant respiratory gas flow via the connecting line 40 by means of the metering device 20. For this purpose, the metering device 20 produces continuously gas pulses with a pulse frequency which is at least independent fromthe respiratory rate of the patient.

In addition, a measurement line 41 is connected to the patient feed line 30 and conducts at least some of the gas mixture situated in the patient feed line 30 to the connector A of the metering device 20. The gas mixture supplied to the metering device 20 via the connector A is analyzed by a measurement/evaluation unit 44 of the metering device 20.

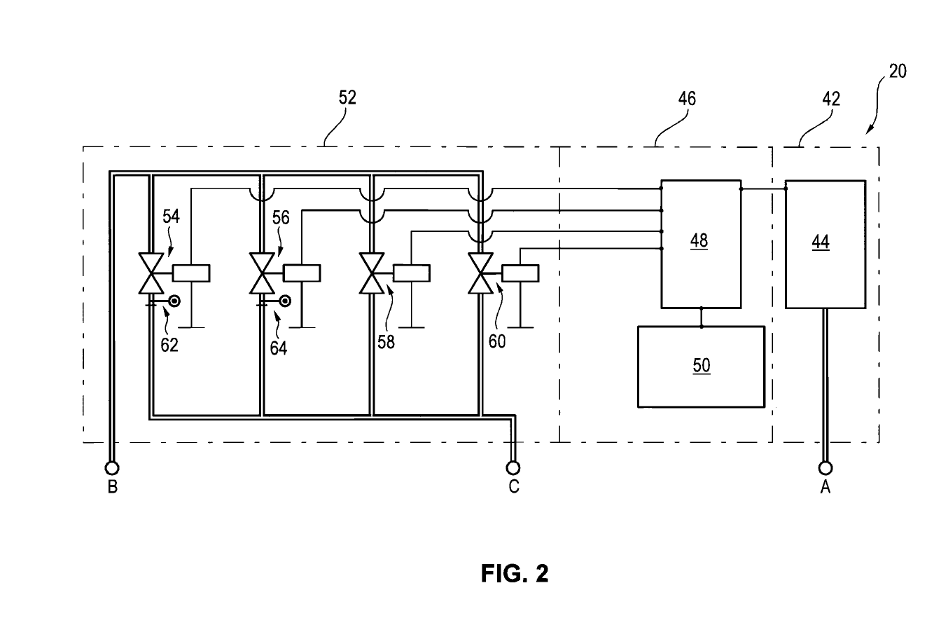

FIG. 2 shows a diagram containing components of the metering device 20 according to FIG. 1. The metering device 20 is also referred to as an NO-administering apparatus because of the nitrogen monoxide used as medical gas in the exemplary embodiment. The metering device 20 has a first module 42 containing a measurement/evaluation unit 44, which analyzes the proportion of NO in the gas mixture (O₂/N₂/NO) supplied via the connector A and transmits a corresponding measured value to a control unit 48 arranged in the second module 46. The control unit 48 is connected to an operating unit 50 in the form of a human-machine interface. The operating unit 50 is preferably designed as a touchscreen. Via the operating unit 50, it is possible to set parameters of the metering device 20, more particularly target values. In addition, set values, measured values and operating values can be output via a display unit of the operating unit 50. The control unit 48 is preferably connected to a control unit of the ventilator 12 via a data cable, which is not shown. Via this data cable, relevant parameters measured values and further information can be transmitted, preferably bidirectionally, between the control unit 48 and the control unit of the ventilator 12.

The metering device 20 has a third module 52, which, in the present exemplary embodiment, comprises four solenoid valves 54 to 60, which are each supplied with the medical gas mixture NO/NO₂ via the connector C. Upstream of the solenoid valves 54, 56 is, in each case, a metering orifice 62, 64 for restricting the flow through the respective solenoid valve 54, 56. The output sides of the solenoid valves 54 to 60 are connected to the connector B, and so the solenoid valves 54 to 60 are connected in parallel. The control unit 48 of the metering device 20 can individually control the solenoid valves 54 to 60, i.e., open them individually or in combination. Thus, it is possible to achieve a gas flow between the connector C and the connector B by opening a valve 54 to 60 and to thus feed medical gas NO via the connecting line 40 into the respiratory gas line 24. The amount of flow between the connector C and the connector B can be increased by the simultaneous opening of multiple valves 54 to 60. In addition, the administered amount, i.e., the amount of the medical gas NO fed into the respiratory gas line 24, can be set by appropriate selection of the pulse duration and/or by appropriate selection of the pulse frequency. In this connection, the gas pulses produced by the individual valves 54 to 60 can have a different pulse duration with preferably the same pulse frequency. The third module containing the parallel arrangement of multiple valves 54 to 60 is also referred to as valve bank 52. The valve bank 52 containing the four solenoid valves 54 to 60 allows a large adjustable metering range and flexible adaptation of the amount of gas to be administered when using gas sources 16 having different starting concentrations of the medical gas. The starting concentration is preferably preset as a parameter via the operating unit 50 and taken into consideration when calculating the pulse duration and pulse frequency for producing the amount to be administered.

In the present exemplary embodiment, the solenoid valve 54 has a flow of 0.16 liters per minute, the solenoid valve 56 has a flow of 1.6 liters per minute and the solenoid valves 58 and 60 each have a flow of 8 liters per minute, measured using medical air. The pulse frequency, i.e. the clock rate, amounts to 104 gas pulses per minute, i.e. 104 bolups per minute. If smaller quantities of the medical gas NO shall be administered or for other reasons a lower clock rate and/or a lower pulse frequency shall be selected, said pulse frequency is preferably reduced to 52 gas pulses per minute or 26 gas pulses per minute. If a higher pulse frequency shall be selected, said pulse frequency can also be increased to 208 gas pulses per minute.

By means of the arrangement shown in FIG. 1, maximum starting doses of 40 ppm are administered in the case of adult patients and maximum starting doses of 20 ppm are administered in the case of children. In the case of newborn babies or premature babies, the maximum starting dose can be lower.

To wean the patient, the dose is lowered in a stepwise or continuous manner to 0.5 ppm; in the case of premature babies, to 0.1 ppm. The starting concentration of the medical gas in the gas source 26 is preferably 1000 ppm. All doses indicated refer to the respiratory air supplied to the Y-piece 26 and containing the introduced medical gas.

In general, the use of a valve bank 52 containing multiple valves 54 to 60 arranged in parallel makes it possible, in the case of currently conventional administered amounts, to use gas sources 16 containing higher starting concentrations of the medical gas, more particularly up to 2000 ppm or up to 4000 ppm. Compared to gas sources containing 1000 ppm of the same amount of gas, the service lives are doubled when the starting concentration is doubled. Alternatively or additionally, the use of the valve bank 52 provides a larger therapeutic concentration spectrum. In the present exemplary embodiment, the minimum opening duration of the solenoid valves 54 to 60 is 7 milliseconds. As a result, the amount of the medical gas NO fed into the respiratory gas line 24 can be varied in large ranges, resulting in a large adjustable therapeutic concentration spectrum.

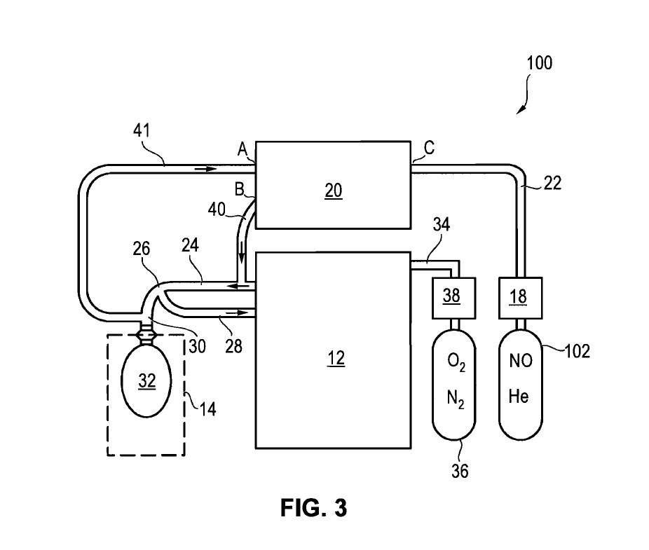

FIG. 3 shows a diagram of a device 100 for administering at least one medical gas to a patient mechanically ventilated by means of a ventilator 12 according to a second exemplary embodiment of the invention. The device 100 matches the device 10 according to FIG. 1 in terms of structure and function. In contrast to FIG. 1, the medical gas nitrogen monoxide (NO) is provided as a gas mixture comprising nitrogen monoxide (NO) and helium (He). Preferably, the gas mixture (NO/He), apart from customary impurities, consists of nitrogen monoxide (NO) and helium (He). This gas mixture (NO/He) is provided by means of a gas source 102 in the form of a gas cylinder and supplied to the metering device 20 via the pressure regulator 18 and the connecting line 22 in the connector C. The gas mixture (NO/He) composed of nitrogen monoxide (NO) and helium (He) achieves very short response times. The gas pulses produced are immediately fed into the respiratory gas line 24.

It was found in experiments that the use of a gas mixture (NO/He) composed of nitrogen monoxide and helium, compared with the gas mixture (NO/N₂) used in the first exemplary embodiment according to FIG. 1 and composed of nitrogen monoxide and nitrogen, achieves a lower compression of the gas mixture (NO/He) composed of nitrogen monoxide and helium and thus achieves more direct feeding of the gas pulse into the respiratory air feed line. As a result, a corresponding pulse-like partial pressure increase is also measurable at the patient 14, and so in particular the pulse frequency of the gas pulses is perceptible by the patient 14. In the exemplary embodiment according to FIG. 1, a partial pressure increase brought about by the gas pulses is also measurable at the patient 14. However, in the case of identical gas pulses, the rise in the partial pressure at the patient 14 and the drop in the partial pressure after a gas pulse steeper when using the gas mixture NO/He than when using the gas mixture NO/N₂.

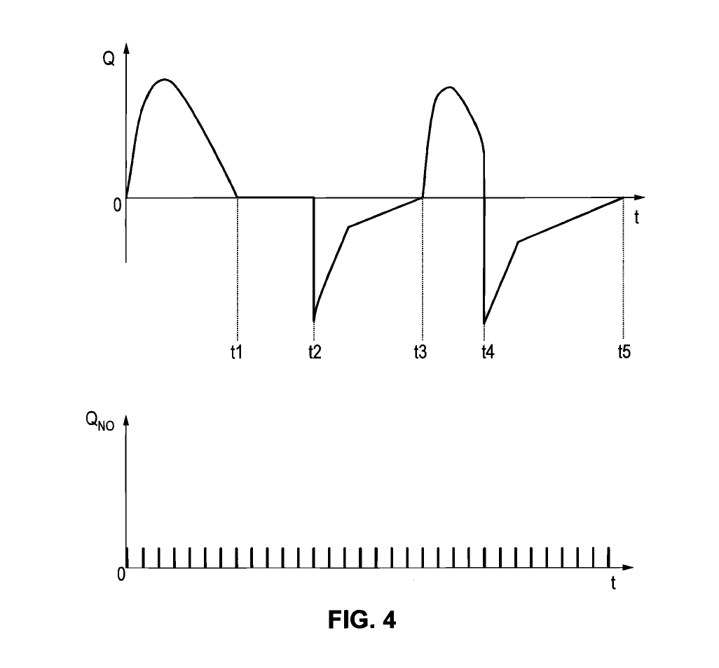

FIG. 4 shows representations of the temporal courses of the respiration of the mechanically ventilated patient 14 and the administration of the medical gas in the form of gas pulses. The upper graph shows the temporal course of the respiration of the patient 14 as volume flow Q. In the period between t0 and t1, a first inhalation phase of the patient 14 takes place. In the period between the times t1 and t2, apnea of the patient 14 occurs. Between the time t2 and t3, a first exhalation phase of the patient 14 takes place and, between the times t3 and t4, a second inhalation phase takes place which is shorter compared to the first inhalation phase. Between the times t4 and t5, a second exhalation phase takes place.

The second, lower graph shows the gas pulses fed into the respiratory air feed line 24 by means of the metering device 20 as volume flow of the relevant proportion of the medical gas NO. Supplying the medical gas in this exemplary embodiment is achieved by means of gas pulses having a constant pulse frequency and thus independently of the respiratory rate of the patient 14.

The solenoid valves used are preferably valves switchable between a completely closed and a completely opened position, which valves are controlled in a binary manner.

The invention can be used especially in neonatology for treating pulmonary hypertension of a premature baby with nitrogen monoxide. Nitrogen monoxide is also administered in order to treat patients after organ transplantations. However, the devices 10, 100 described in the exemplary embodiments can also be used for administering other gaseous medicaments.

It is further known to mix gaseous medicaments into a respiratory gas flow by means of a proportioning valve as a function of the flow velocity, measured in real-time by means of a flow meter, of the respiratory air flow.

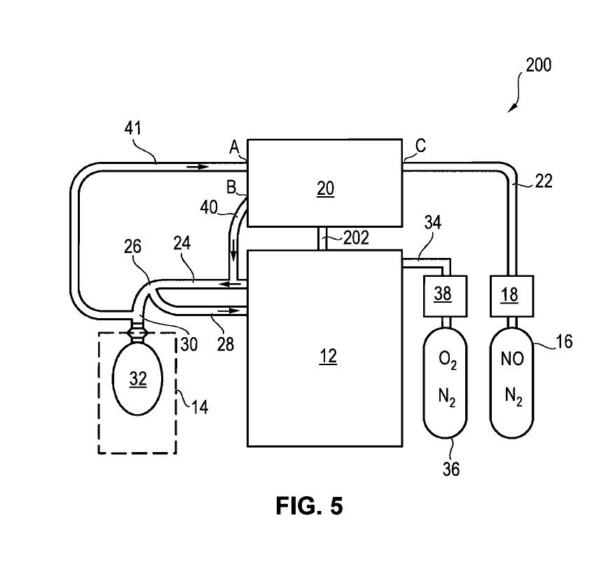

FIG. 5 shows a diagram of a further device 200 for administering at least one medical gas to a patient 14 mechanically ventilated by means of a ventilator 12 according to a third exemplary embodiment of the invention. In contrast to the exemplary embodiments according to FIG. 1 and according to FIG. 3, the medical gas NO is metered into the patient circle part of the ventilator 12, i.e., into the respiratory air tube 24, in proportion to the respiratory course of the patient 14. In contrast to the exemplary embodiments according to FIGS. 1 and 3, in the third exemplary embodiment, gas pulses having different gas volumes are produced as a function of the respiratory phase and/or the course of the respiratory phase.

There is a data and/or signal cable 202 between the ventilator 12 and the metering device 20, via which information concerning a real-time flow profile of the respiration of the mechanically ventilated patient 14 transmits by means of signals and/or data to the control unit 48 of the metering device 20. For the data transmission, it is possible to use in particular a real-time-capable bus system, for example a CAN BUS or a serial interface, such as a USB interface or RS232 interface, using a real-time-capable data transmission protocol.

The medical gas is fed into the respiratory air feed line 24 such that, during the respiratory phases of the patient, a higher concentration of the medical gas is contained in the supplied ventilation air. In a first embodiment of the third exemplary embodiment, the gas pulses are delivered at a constant pulse frequency, wherein the amount of gas delivered per gas pulse is greater during the inhalation phases than during apnea phases and during the exhalation phases of the patient 14.

Alternatively or additionally, it is possible in further embodiments for the pulse frequency to be higher during the inhalation phases than during the exhalation phases and during apnea. In addition, it is possible during apnea of the patient 14 for the supplying of the medical gas by the metering device 20 to be interrupted. It is advantageous that by means of gas-pulse and pulse-frequency optimization performed by the control unit 44 or a control unit of the ventilator 12 a relatively long opening time of the activated valves 54 to 60 is required within the defined pulse frequency. The pulse frequency is preferably 104 gas pulses per minute. Only when the required gas flow of the medical gas through the valve bank 52 is greater than or equal to the maximum flow through a valve 54 to 60, and so the flow through said valve 54 to 60 would not be sufficient to administer the required amount of the medical gas, or the valve 54 to 60 would no longer close and would thus produce no more gas pulses, is an additional further valve 54 to 60 or, instead of the first valve 54 to 60, a second valve 54 to 60 having a larger flow in the opened state controlled by the control unit 48.

In a fourth exemplary embodiment, in contrast to the exemplary embodiment shown in FIG. 5, the medical gas NO is not provided as a gas mixture composed of nitrogen monoxide and nitrogen (NO/N₂), but as a gas mixture composed of nitrogen monoxide (NO) and helium (He). The advantages associated with this gas mixture (NO, He) have already been elucidated in conjunction with FIG. 3. The gas pulses are produced in this fourth exemplary embodiment as described for the third exemplary embodiment in conjunction with FIG. 5.

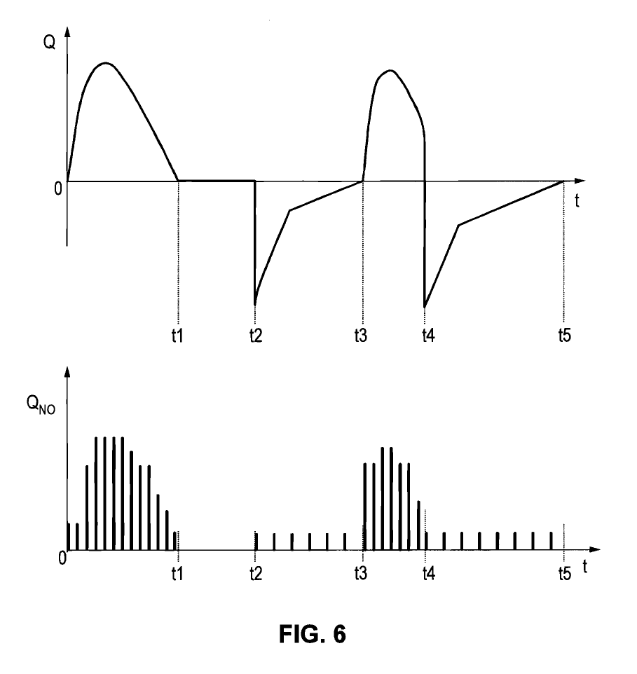

FIG. 6 shows a representation of the temporal course of the respiration of the mechanically ventilated patient 14 and the temporal course of the administration of the medical gas (NO/N₂)/(NO/He). The upper graph shows the respiratory air flow of the mechanically ventilated patient 14, similar to FIG. 4, and the lower graph shows the temporal course of the gas pulses, by means of which the medical gas NO or the gas mixture (NO/N₂), (NO/He) is fed into the respiratory gas feed line 24. In the exemplary embodiment shown, it can be seen that, during the inhalation phases of the patient, the gas flow through the valves 54 to 60 or through the valve bank 52 at constant pulse width is varied by a specific selection and/or combination of different valves 54 to 60.

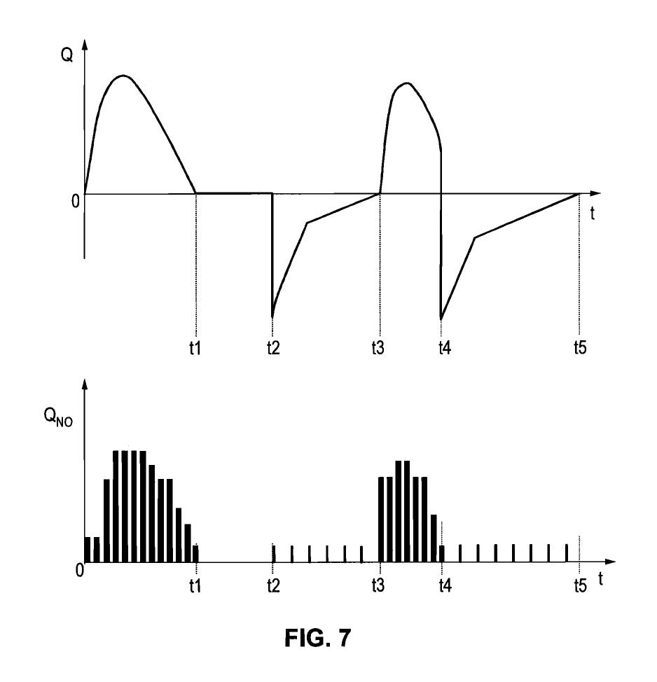

FIG. 7 shows a representation of the temporal course of the respiration of the mechanically ventilated patient 14 and of the administration of the medical gas according to a fourth exemplary embodiment of the invention. The fourth exemplary embodiment differs from the exemplary embodiment shown in FIG. 6 in that gas pulses having a greater pulse width are administered during the inhalation phases than during the exhalation phases. Thus, the administered amount of medical gas is increased. More particularly, what can be achieved by this, even with high flow velocities of the respiratory gas, is that the amount of administered medical gas is proportional to the flow velocity. The pulse widths of at least two gas pulses introduced during one inhalation phase can be different.

In other exemplary embodiments, the amount of gas administered in one gas pulse can be further varied in that the individual pulse widths, with which the valves 54 to 60 for producing a gas pulse are controlled, are different, and so at least two valves 54 to 60 deliver gas pulses of different pulse width. As a result, a total gas pulse is produced which has been produced from two subpulses of different pulse width. The total gas pulse then has a stepped course, which is fed into the respiratory gas feed line 24. In a specific embodiment of the third and fourth exemplary embodiment, the pulse frequencies during the inhalation phases are twice as high as in the exhalation phase. For example, the pulse frequency can be 208 gas pulses per minute during the inhalation phase and 104 gas pulses per minute during the exhalation phase. Alternatively, the pulse frequency can be 104 gas pulses per minute during the inhalation phase and 52 gas pulses per minute during the exhalation phase. Depending on the rise in the amount of gas inhaled at the start of an act of inhalation by the patient 14, i.e., depending on the flow at the start of the act of inhalation and/or the temporal course of the respiratory gas flow, it is possible for the length of an inhalation of the patient 14 and/or the course of the inhalation of the patient 14 to be empirically determined and, in line with the estimated course for each gas pulse during an inhalation, for an amount of the medical gas to be fed into the respiratory gas feed line 24 by this gas pulse to be defined. The defined amount of gas is then fed into the respiratory gas feed line 24 by appropriate control of the solenoid valves 54 to 60.

In an alternative embodiment of the invention, a closed circuit system is formed, and so the gas mixture exhaled by the patient 14 remains in the closed circuit system. Thus, the medical gas not taken up by the patient also remains in the circuit system. Such closed circuits are used especially during anesthesia of the patient 14. During anesthesia, the patient 14 is connected to an anesthesia machine. The control unit 48 is connected to the anesthesia machine via an interface. The anesthesia machine comprises at least one sensor for determining the start of a breath of the patient 14 and a sensor for determining the volume of gas mixture inhaled in said breath. The anesthesia machine transmits, via the interface, data containing information concerning the start of the breath and the inhaled volume of gas mixture to the control unit 48, which, as a function of said data, determines the amount of the medical gas injecting via the valves 54 to 60 such that as much medical gas is injected for it to be completely or at least almost completely taken up by the patient 14 in the breath, and so no accumulation of the medical gas occurs in the gas mixture of the closed circuit system. The control unit 48 controls the solenoid valves 54 to 60 in particular such that the amount of medical gas to be injected is injected within a short time at the start of the breath. Thus, an accumulation of the medical gas in the gas mixture is avoided and, as a result, reactions with other substances in the closed circuit system are, for example, avoided.

In a further alternative embodiment of the invention, the medical gas is taken up in a carrier gas, more particularly helium. This reduces time delays in the transport of the medical gas through the lines, and so a precise control of the inspiration times is possible. This is necessary especially in the treatment of infants, since, during their treatment, even delays of 100 ms in the inspiration times may be critical with respect to the success or failure of the therapy. The ventilator 12 comprises a sensor for calculating the gas volume of a breath of the patient 14 and a sensor for determining the temporal start of a breath. The ventilator 12 is connected to the metering device 20 via a data interface, wherein data containing information concerning the volume of the last breath of the patient 14 and data containing information concerning the times of at least the last two breaths of the patient 14 are transmitted via the interface. The control unit 48 determines in real-time, as a function of these data, the start of the next breath of the patient 14 and controls, as a function of the calculated start of the breath and of at least the gas volume of the last breath, the solenoid valves 54 to 60 such that the injecting amount of medical gas is injected in a burst at the start of the next breath. Injection in a burst is understood to mean in particular that the medical gas is injected within a very short time. For this purpose, the control unit 48 opens the solenoid valves 54 to 60 as far as possible at the start of the breath.

Although the invention above has been described in connection with preferred embodiments of the invention, it will be evident for a person skilled in the art that several modifications are conceivable without departing from the invention as defined by the following claims.

What is claimed is:

1. A method for administering at least one medical gas to a patient mechanically ventilated by means of a ventilator,
in which a first end of a first line supplying at least respiratory gas from the ventilato, a first end of a second line discharging at least exhaled gas from the patient and a first end of a patient feed line are connected to one another via at least one connecting piece,
by means of the ventilator at least in one portion of the first line a respiratory gas flow is produced, and
in which the medical gas is introduced into the first line supplying the respiratory gas,
wherein a gas source for providing the medical gas to be administered and the first line are connected via at least two regulating means arranged in parallel, wherein a connection is established between the gas source and the first line via each regulating means in the opened state,
in that multiple gas pulses of the medical gas are fed successively into the first line by means of the regulating means, and in that the gas pulses depending on at least one parameter of the respiration of the patient are fed.

2. The method according to claim 1, wherein the flow rate of the respiratory gas flow is used as a parameter for the respiration of the patient and in that the respective pulse duration of the gas pulse and/or the respective volume flow of the medcial gas during the gas pulses and/or the interval between consecutive gas pulses are controlled depending on the flow rate and/or the gradient of the flow rate.

3. The method as claimed in claim 1, wherein a pulse frequency of the gas pulses at least during an interval is 26, 52, 104 or 208 pulses/minute.

4. The method as claimed in claim 1, wherein the regulating means are controlled by means of a control unit such that an amount of gas defined in relation to a gas pulse is fed into the first line.

5. The method as claimed in claim 1, wherein the medical gas contains NO, preferably NO and N₂ or NO and He.

6. The method as claimed in claim 1, wherein there are more than two, preferably four, regulating means.

7. The method as claimed in claim 1, wherein the regulating means in the opened state allow volume flows which are different from one another to pass through from the gas source to the first line.

8. The method as claimed in claim 1, wherein the regulating means each comprise a solenoid valve.

9. The method as claimed in claim 1, wherein at least one restricting orifice for limiting the volume flow flowing through the regulating means is arranged upstream and/or downstream of at least one regulating means.

10. The method as claimed in claim 1, wherein regulating means of the same type are used and in that the volume flow flowing through the regulating means in the opened state differs owing to the provision of different flow resistances.

11. The method as claimed in claim 1, wherein the regulating means are switched between a completely opened state and a completely closed state.

12. The method as claimed in claim 1, wherein gas is removed from the patient feed line and in that at least the proportion of the medical gas in the removed gas is determined.

13. The method as claimed in claim 12, wherein the gas is removed from the patient feed line via a measurement line and supplied to an analysis unit for the detection of at least the proportion of the medical gas.

14. The method as claimed in claim 12, wherein the determined proportion of the medical gas, as the actual value, is compared with a target value, and in that, in the event of a determined deviation of the actual value from the preset target value, the amount of the medical gas introduced into the first line during the gas pulse is adapted as a function of the comparative result, preferably regulated to the target value.

15. The method as claimed in claim 12, wherein at least the proportion of a reaction product of the medical gas, preferably an oxidation product of the medical gas, is detected, wherein the medical gas is preferably NO and the oxidation product is NO₂.

16. The method as claimed in claim 1, wherein the ventilator determines information concerning a flow profile of the respiration of the mechanically ventilated patient and in that depending on the determined flow profile a control unit controls the regulating means such that during the inhalation phases of the patient at each generated gas pulse a larger amount of the medical gas (NO) is supplied into the first line than is the case during the exhalation phases of the patient.

17. The method as claimed in claim 16, wherein during the exhalation phase the pulse frequency is constant or that during the inhalation phase the pulse frequency is higher than is the case during the exhalation phase.

18. The method as claimed in claim 16, wherein between the inhalation phase and the exhalation phase an there is an apnea phase and in that during this apnea phase no medical gas is supplied.

19. The method as claimed in claim 16, wherein during the inhalation phase gas pulses having a larger pulse width than during the exhalation phase are generated.

20. A device for administering at least one medical gas to a patient mechanically ventilated by means of ventilator,
having a first line supplying at least respiratory gas from the ventilator,
having a second line discharging at least exhaled gas from the patient,
having a patient feed line, wherein a first end of the first line, a first end of the second line and a first end of the patient feed line are connected to one another via at least one connecting piece,
having a ventilator which produces at least in one portion of the first line a constant respiratory gas flow, and
having supply means for supplying the medical gas to the first line supplying the respiratory gas,
wherein there are at least two regulating means arranged in parallel which connect a gas source for providing the medical gas to be administered and the first line, wherein each regulating means in the opened state establishes a connection between the gas source and the first lin, and
having a control unit which controls the regulating means such that at least one regulating means successively feeds multiple gas pulses of the medical gas into the first line, and
in that the control unit depending on at least one parameter of the respiration of the patient controls the regulating means .

21. The device as claimed in claim 20, wherein, as a function of the amount of the medical gas to be introduced into the first line, the control unit selects a regulating means to be opened for producing the gas pulse or selects multiple regulating means to be opened, and the pulse duration defines, as a function of the pressure difference present between a feed line of the gas source and the first line, gas flow to be expected in the case of the selected regulating means or in the case of the multiple selected regulating means.
